(19) United States
(12) Patent Application Publication
(10) Pub. No.: US 2012/0264210 A1
(43) Pub. Date: Oct. 18, 2012

(54) Method for Expanding and/or Preserving Cells by Means of Gas Enrichment of the Culture Medium

(75) Inventors: Pierre-Eloi Bontinck, Lille (FR); Arnaud Chavatte, Isbergues (FR); Cecile Coissac, Lille (FR)

(73) Assignee: MACO PHARMA, Mouvaux (FR)

(21) Appl. No.: 13/505,548

(22) PCT Filed: Nov. 2, 2010
(86) PCT No.: PCT/FR10/00725

§ 371 Date: May 2, 2012

(30) Foreign Application Priority Data
Nov. 3, 2009 (FR) 0905274

(51) Int. Cl. C12N 5/071; C12M 1/36; C12M 3/00

(52) U.S. Cl. 435/366; 435/303.1; 435/286.6

(57) ABSTRACT

A method for expanding and/or preserving cells inside a culture vessel containing a culture medium provides for the renewal of the culture medium by a culture medium stream, at least a portion of which comes from a culture medium volume contained in a gas-enrichment vessel of the culture medium, the enrichment-vessel further containing a gas volume which is separated from the culture medium volume by a free interface, the gas volume being renewed by a gas stream that is introduced into the enrichment vessel directly into the gas volume, the flow of the stream being arranged so as to enable a gas exchange between the culture medium volume and the gas volume at the interface thereof.

### BACKGROUND

The invention relates to a method for expanding and/or preserving cells, a kit for implementing this method as well as an apparatus in which said kit is intended to be used.

The invention applies to the ex vivo expansion and/or preservation of cells, in particular cells used in cellular therapy.

The advent of in vitro cell cultures that can be transplanted directly into humans is at the origin of the development of different types of bioreactors for packaging said cultures.

One of the important parameters for the culture or preservation of these cells is the supply of oxygen. A number of methods have been developed for supplying oxygen to the cells.

For example, document EP 1 935 974 envisaged a closed culture container produced with a film having high permeability to oxygen and carbon dioxide.

In document US 2008 012894, the culture medium is oxygenated by means of a membrane oxygenator before being introduced into a bioreactor.

In these two documents, the transfer of oxygen is performed through a film or a membrane, which limits the rate of oxygen transfer to the liquid.

Alternatively, document US 2005 0032208 proposes supplying the bioreactor with a culture medium that has previously been aerated. The aeration is performed by means of a bottle equipped with an air inlet tube immersed in the medium and an air outlet tube for removing air from the bottle.

This aeration system has the disadvantage of generating bubbles in the culture medium, causing foam to form. In addition, the bottle must be connected to the rest of the system at the time of use, which results in a loss of time, and does not facilitate in the use of the system and presents a risk of contamination.

Document U.S. Pat. No. 6,190,913 also proposed placing the bioreactor on an agitator in order to improve the transfer of oxygen contained in the bioreactor. However, the cells arranged in this bioreactor are subject to stress, which can cause damage to the cells.

### SUMMARY OF THE INVENTION

The invention is intended to overcome these disadvantages by providing a simple system for oxygenation of a culture medium, which can be pre-connected during manufacture to the culture container in order to form a closed system. In addition, the oxygenation is performed directly on the culture medium, outside the culture container, so as not to disturb the cells.

To this end, the invention proposes a method for expanding and/or preserving cells inside a culture container containing a culture medium, in which said method involves renewing said culture medium by means of a stream of culture medium of which at least a portion comes from a culture medium volume contained in a container for gas enrichment of said culture medium, in which said enrichment container also contains a gas volume that is separated from the culture medium volume by a free interface, with the gas volume being renewed by means of a gas stream that is introduced into the enrichment container directly in the gas volume, with the flow of said stream being arranged so as to enable a gas exchange between said culture medium volume and said gas volume at the interface thereof.

According to a second aspect, the invention relates to a kit for implementing the method according to the first aspect of the invention, in which the kit includes a culture container intended to contain cells in a culture medium and a container for gas enrichment of the culture medium, in which said enrichment container has a supply opening for the culture medium to be enriched and a withdrawal opening for the enriched culture medium, in which said withdrawal opening is in fluidic communication with a supply opening of the culture medium, said enrichment container also has a supply opening and a withdrawal opening for gas, in which said openings of the enrichment container are arranged so that said container can contain a culture medium volume into which the culture medium supply and withdrawal openings lead, and a gas volume into which the gas supply and withdrawal openings lead.

A third aspect of the invention concerns an apparatus in which a kit according to the second aspect of the invention is intended to be used, including:
means for attaching the enrichment container by arranging the gas volume above the culture medium volume;
means for receiving the culture container, a possible collection container and a possible culture medium source;
a device for circulating the gas contained in the enrichment container, in which said device includes a pump of which the outlet is placed in communication with the gas supply opening of the enrichment container and the inlet is in communication with a gas source, in which said device enables gas to be introduced into the enrichment container with a flow arranged so as to enable a gas exchange between said culture medium volume and said gas volume at the interface thereof;
a device for circulating the culture medium stream between the enrichment container, the culture container, a possible collection container and a possible culture medium source.

### BRIEF DESCRIPTION OF THE DRAWINGS

The invention can be better understood in light of the following description.

FIG. 1 shows a main diagram of the method according to the invention.

FIG. 2 diagrammatically shows a kit intended for the expansion and/or preservation of cells according to the invention.

FIG. 3 diagrammatically shows a front view of the apparatus for implementing the method according to the invention with an enrichment container.

FIGS. 4 and 5 diagrammatically show a perspective view of the apparatus of FIG. 3 with the upper plate in the top position and in the bottom position, respectively.

### DETAILED DESCRIPTION OF THE PREFERRED EMBODIMENT(S)

The invention relates to the expansion and/or the preservation of cells, in particular human cells for therapeutic use.

To enable cell development, i.e. their survival as well as their proliferation and/or their differentiations, the cells are incubated in a container containing a culture medium under standard conditions. The standard conditions for human cells are, for example, a temperature of around 37° C. in an atmosphere controlled at 5% CO₂ at a pH of around 7. These standard conditions are obtained by placing the culture container in an incubator-type apparatus.

The culture medium contains the different elements necessary for cell growth. The culture medium includes in particular a commercially available base medium such as Dulbecco's Modified Eagle's Medium (DMEM), Minimal Essential Medium (MEM), RPMI1640, DMEM/F-10 or DMEM/F12. This base medium may contain other elements such as growth factors.

All of the cell types require, in addition to the culture medium, oxygen in order to develop. The method of the invention enables the culture medium to be enriched with gas and in particular oxygen in order to provide the cells with this element, which is essential for their development.

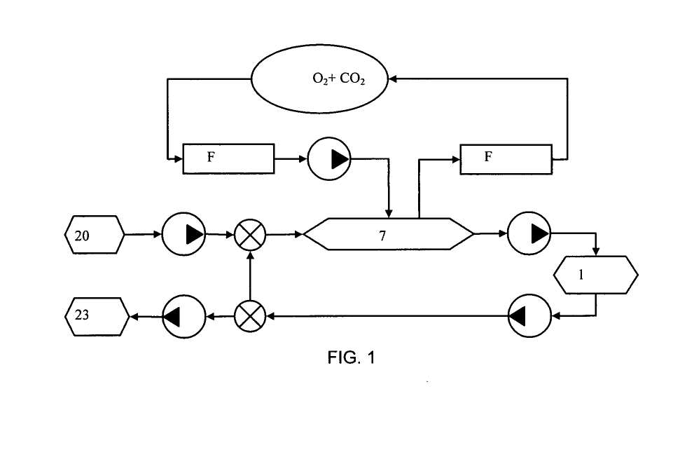

According to a first aspect and in relation to FIG. 1, the invention proposes a method for the expansion and/or preservation of cells inside a culture container 1 containing a culture medium by means of a culture medium stream.

The cells are developed in a culture medium stream, i.e. the culture medium is renewed by means of a culture medium stream. The stream is continuous or discontinuous. For example, the culture medium is renewed at regular or irregular intervals. The cells are either adherent cells, i.e. they adhere to the culture medium, or non-adherent cells. In this latter case, the cells are suspended in the culture medium. To cultivate them under flow conditions, they must be heavy enough not to be driven into the stream of culture medium and/or the stream must be arranged so as not to drive them.

At least some of the culture medium in the culture container 1 comes from a culture medium volume contained in a container 7 for gas enrichment of the culture medium.

The gas enrichment is performed in the enrichment container 7 that includes a gas volume and a culture medium volume, the gas and culture medium being separated by a free interface where a gas exchange takes place.

The gas comes from a gas source such as a bottle of oxygen O₂ or an oxygen and carbon dioxide mixture. Alternatively, the gas source is ambient air or the air of an incubator when the cells are cultivated in an incubator.

The culture medium and the gas are therefore in direct contact with one another. In particular, the enrichment container 7 does not include a porous membrane or semi-permeable film separating the gas from the culture medium. The lack of barrier between the two gas and culture medium volumes facilitates gas exchanges between said volumes.

According to the invention, the gas volume is renewed in the enrichment container 7 by means of a gas stream, i.e. the gas circulates inside the enrichment container 7. The gas volume moves above the culture medium volume.

The gas stream is introduced in the enrichment container 7 directly into the gas volume. The gas is not introduced directly into the culture medium volume so as to prevent the formation of gas bubbles in the culture medium.

Indeed, the presence of certain substances in the culture medium, such as albumin, has a tendency to cause the culture medium to foam when it is agitated or splashed around.

The gas stream produced outside the culture medium therefore prevents the formation of foam in the culture medium during its enrichment with gas.

The gas exchange is in particular performed by diffusion of the gas in the culture medium along the free interface between the gas and the culture medium.

According to the invention, the gas stream is arranged so as to improve the gas exchange between the culture medium volume and the gas volume, and in particular so as to improve the diffusion of gas in the culture medium volume.

To do this, the gas stream is arranged so that the pressure in the enrichment container 7 is greater than the ambient pressure.

The gas stream is forced and circulated in the enrichment container 7 along a flow path, for example by means of a pump. The gas introduced is thus pressurised.

The gas exchange is also improved by supplying the enrichment container 7 with a suitable culture medium stream, for example by means of a pump. This pump makes it possible in particular to isolate the culture cells from the pressure present in the enrichment container. The cells thus remain at ambient pressure.

According to an alternative, the gas stream has a direction of flow that is opposite that of the culture medium stream. Under certain conditions, these opposite streams enable better diffusion of the gas in the culture medium.

The pressure of the gas inside the enrichment container 7 is greater than the ambient pressure. This pressure is caused, for example, by the pressurised introduction of the gas into the enrichment container 7 and the creation of a pressure drop at the outlet of said container.

The pressure drop at the outlet of the enrichment container 7 is created by the passage of the gas into an obstacle, such as a narrowing and/or a restriction of the cross-section, such as a porous filter F.

The gas stream flows into the enrichment container 7 in a flow path having a portion for supplying gas to the container and a portion for removing gas from the container.

To pressurise the gas volume in the enrichment container 7, the gas is introduced under pressure into the container 7 by said supply portion, and the withdrawal portion has a pressure drop that is designed to maintain a pressure higher than the ambient pressure in the enrichment container 7.

The pressure drop of the withdrawal portion must be great enough and depends upon the desired pressure for the enrichment container. For example, a significant narrowing of the cross-section at the withdrawal portion makes it possible to impose a gas pressure in the enrichment container 7 that is greater than the ambient gas pressure. To do this, the gas pressure at the supply portion must be high enough.

More concisely, the greater the pressure drop is, the higher the pressure in the enrichment container 7 will be, within the pressure limit of the pump and to prevent said container from exploding.

According to an embodiment, the culture medium stream supplying the enrichment container 7 comes at least partially from the culture container 1. In this embodiment, the culture medium of the culture container 1, in which oxygen has been used by the developing cells, is recycled in the enrichment container 7 so as to again be enriched with gas. This recirculation step makes it possible to reduce the consumption of culture medium.

According to another embodiment, the culture medium stream comes at least partially from a fresh culture medium source 20, i.e. a culture medium that has not yet been used.

These two embodiments may be combined in order to supply the enrichment container 7 partially with culture medium coming from the culture container 1 and partially with fresh culture medium. The cells of the culture container 1 are then expanded and/or preserved in a partially fresh and gas-enriched and partially recycled medium providing them with sufficient nutrition and oxygen for their development, while reducing the waste produced by the cells and the consumption of fresh medium.

When the culture medium stream is not recycled, it is withdrawn from the culture container so as to be stored, in particular in a collection container 23 intended to be thrown away.

The culture container 1 is intended to contain cells in a culture medium. It may be solid or rigid, in particular in the form of a bag formed, for example, by assembling two flexible sheets welded at their periphery so as to form a variable internal volume.

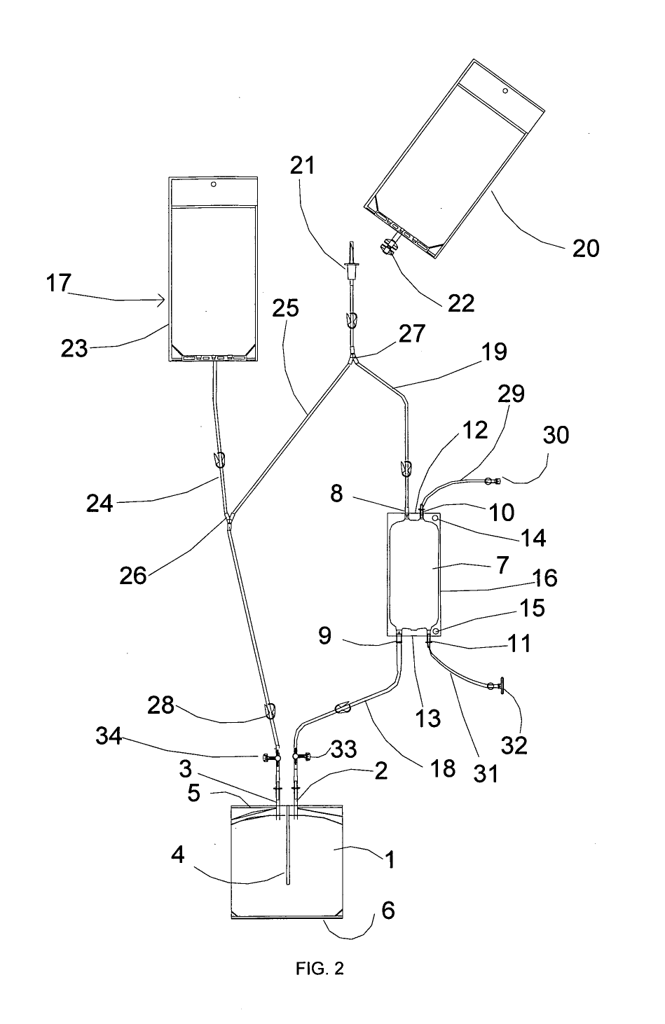

The culture container 1 shown in FIG. 2 has a culture medium supply opening 2 and a culture medium withdrawal opening 3.

The supply and withdrawal openings 2, 3 of the culture container 1 are, for example, tubular portions.

To improve the circulation of the culture medium in the culture container 1, the container includes at least one deflector 4 arranged on the direct flow path between the supply opening 2 and the withdrawal opening 3 for the culture medium of said culture container.

In FIG. 2, the culture container 1, hereinafter referred to as the culture bag, contains a deflector 4 made of a weld extending from an edge 5 of the bag to near the opposite edge 6, leaving a passage for the circulation of the culture medium. The edge 5 from which the weld extends includes culture medium supply and withdrawal openings 2, 3, the weld being arranged between the two openings. The weld is substantially parallel to the two other edges of the bag.

In particular, the peripheral welds of the culture bag 1 are round so as to facilitate the flow of medium in the bag without creating turbulence.

The enrichment container 7 may be in rigid or semi-rigid form. According to FIG. 2, it is in the form of a flexible envelope defining a variable volume. This envelope, hereinafter referred to as the enrichment bag 7, is formed by the assembly, for example at their peripheries, of two flexible sheets so as to form a variable internal volume. When, according to the method of the invention, the pressure inside the enrichment bag 7 is greater than the ambient pressure, the bag swells.

The enrichment container 7 has an opening 8 for supplying culture medium to be enriched and an opening 9 for removing enriched culture medium. The container 7 also includes a gas supply opening 10 and a gas withdrawal opening 11. Said openings 8, 9, 10, 11 of the enrichment container 7 are arranged so that said enrichment container may contain a culture medium volume into which the culture medium supply and withdrawal openings 8, 9 lead and a gas volume into which the gas supply and withdrawal openings lead.

The openings 8, 9, 10, 11 of the enrichment bag 7 are in particular in the form of tubular portions that lead into the variable internal volume of said bag.

In FIG. 2, the culture medium and gas supply openings 8, 10 are arranged on one side 12 of the enrichment container, and the culture medium and gas withdrawal openings 9, 11 are arranged on the opposite side 13 of said container 7.

In addition, the gas supply and withdrawal openings 10, 11 are arranged above culture medium supply and withdrawal openings 8, 9.

This particular arrangement makes it possible to create a gas stream and a culture medium stream that flow in the same direction.

In addition, the enrichment container 7 is equipped with suspension means enabling said container to be arranged with the gas volume above the culture medium volume.

According to FIG. 2, the bag 7 is equipped with two holes 14, 15 on the side 16 perpendicular to the sides 12, 13 containing the openings 8, 9, 10, 11 and located near the gas supply and withdrawal openings 10, 11. These holes enable it to be suspended in the vertical plane, for example by means of hooks. Once suspended in the vertical plane, the gas supply and withdrawal openings 10, 11 are placed above the culture medium supply and withdrawal openings 8,9. With the enrichment bag 7 in this position, the risk of culture medium leaking through one of the gas openings 10, 11 is reduced.

The culture and enrichment containers 1, 7 are in particular made of ethylene vinyl acetate. They can be sterilised, for example, by beta or gamma radiation.

According to the invention and as shown in FIG. 2, the kit 17 includes a culture container 1 and an enrichment container 7 of which the opening 9 for removing enriched medium is in fluidic communication with the culture medium supply opening 2 of the culture container 1.

In particular, the opening 9 for removing the medium from the enrichment container is connected to the medium supply opening 2 of the culture container by means of a first tubing 18.

In addition, the kit 17 includes means for establishing fluidic communication between the culture medium supply opening 8 of the enrichment container 7 and a fresh culture medium source 20. The fresh medium source 20 may be a flask or a bag of culture medium.

In particular, the culture medium supply opening 8 of the enrichment container 7 is in fluidic communication with a fresh medium source 20, by means of a second tubing 19 connected directly to a fresh medium source or intended to be connected to a fresh medium source.

In this latter case, the means for establishing fluidic communication between the enrichment container 7 and the fresh medium source 20 includes means for connection to the source such as a perforator 21.

For example, the end of the second tubing 19 is equipped with a perforator 21 intended to be engaged in the inlet opening 22 of a bag 20 containing fresh medium. The connection is performed under a suction hood in order to avoid any contamination.

The withdrawal opening 3 of the culture container 1 is in fluidic communication with the supply opening of a collection container 23 intended to collect the used medium coming from the culture container 3, in particular by means of a third tubing 24.

Advantageously, the collection container 23 is in the form of a collection bag formed, for example, by assembling two flexible sheets defining a variable volume. The collection container 23 is equipped with at least one supply opening.

To enable the recirculation or recycling of the culture medium through the gas enrichment container 7, the culture medium withdrawal opening 3 of the culture container 1 is in fluidic communication with the culture medium supply opening 8 of the enrichment container 7.

According to FIG. 2, a fourth tubing 25 is connected between the second and the third tubing 19, 24 by means of two three-way Y- or T-type connectors 26, 27.

Thus, the fluidic communication between the containers 1, 7, 23, 20 is achieved by means of tubings 18, 19, 24, 25 connected to the openings of the different containers and connected to one another.

According to a particular embodiment, the tubings comprise tubing portions intended to be arranged in a pump head of a peristaltic pump. In particular, the hardness of these portions intended to be placed in the pump head is lower than that of the other portions of the tubing, for example a hardness of between 60 and 70 shore A, and in particular 65 shore A.

In FIG. 2, the first tubing 18 between the culture bag 1 and the enrichment bag 7, the second tubing 19 between the enrichment bag 7 and a connector 27 with the fourth tubing 25, the third tubing 24 between the culture bag 1 and the other connector 26 with the fourth tubing 25 and between the collection bag 23 and this other connector 26 include tubing portions intended to be inserted into a pump head.

The kit 17 also includes one or more selective means for opening/closing the flow of fluids in the tubings such as clamps 28.

To enable the establishment of a gas stream in the gas enrichment container 7, the gas supply opening 10 is in fluidic communication or is intended to be in fluidic communication with a pressurised gas source by means of a fifth tubing 29. The pressurised gas source is for example pumped ambient air or a bottle of pressurised gas. The gas may be pure oxygen or a gas mixture such as an oxygen and carbon dioxide mixture.

More specifically, one of the ends of the fifth tubing 29 is connected to the gas supply opening 10 of the enrichment container and the other end is connected or intended to be connected to a pressurised gas source. For example, this other end includes a Luer connector 30.

In particular, the Luer connector 30 is intended to be connected to the outlet opening of a membrane pump so as to introduce pressurised gas into the enrichment container 7. Optionally, means for filtering the gas flow, such as a porous filter F, are arranged at the outlet of said pump so as to filter the gas entering the kit. This filter is advantageously a sterilising filter including a membrane with a porosity of less than 2 μm, in particular 1.2 μm and more particularly 0.22 μm. In this case, the other end of the fifth tubing 29 is connected or intended to be connected to the outlet of the filter F.

The gas withdrawal opening 11 of the enrichment container 7 is connected to the end of a sixth tubing 31. This sixth tubing leads into the ambient air or into a gas collection container and its free end is connected or intended to be connected to gas stream filtration means F such as a porous vent or filter 32. For example, the end of the sixth tubing 31 is equipped with a filter F. In particular, the filter is a sterilising filter having a porosity of less than 2 μm, and in particular around 1.2 μm. It may be made of nylon.

This filter 32 has a double function. First, it prevents contaminants from entering the kit, then it constitutes means for creating a pressure drop at the outlet of the enrichment container 7.

According to an embodiment, the kit 17 also includes means for accessing the interior of the kit. In particular, the kit includes a first three-way valve 33 near the medium supply opening 2 of the culture container 1 intended to introduce the cells into said culture container.

The kit also includes a second three-way valve 34 near the medium withdrawal opening 3 of the culture container 1 intended to withdraw the cells from the culture container.

These three-way valves 33, 34 include in particular means for connection to a syringe.

The kit may include other access means in order to perform, for example, a sampling during culturing.

The kit is advantageously intended for a single use. Intended for clinical use, it must be sterilised. As an example, the kit is sterilised and packaged in a sterile packaging.

In a particular example, the sterile packaging includes the kit consisting of the enrichment bag 7, the culture bag 1 and optionally the collection bag 23, with these bags being pre-connected to one another by means of tubings.

In another example, the packaged kit also includes a vent placed at the end of the sixth tubing 31.

The kit is intended to be used on an apparatus implementing the method according to the invention.

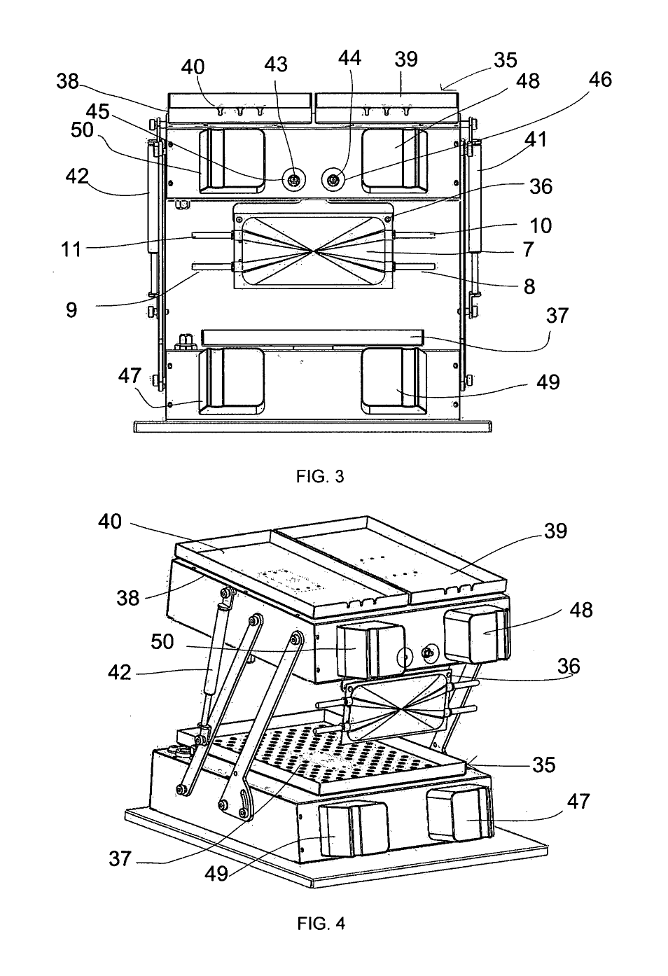

For this and in relation with FIGS. 3 to 5, the apparatus 35 includes means for attaching the enrichment container 7 by arranging the gas volume above the culture medium volume. The medium volume is also kept below gas supply 10 and withdrawal 11 openings.

These means are, for example, in the form of one or more suspension hooks 36 on which the enrichment bag 7 is suspended by means of holes 14, 15 provided for this purpose. The enrichment bag 7 is then placed in the vertical plane, the culture medium supply and withdrawal openings 8, 9 arranged on opposite sides lead into the culture medium volume, and the gas supply and withdrawal openings 10, 11 lead into the gas volume.

The apparatus also includes means for receiving the culture container 1, a possible collection container 23 and a possible culture medium source 20.

In particular, the receiving means include a lower plate 37 and an upper plate 38. The lower plate 37 is intended to receive the culture container 1 and the upper plate 38 is intended to receive the culture medium source 20 and the collection container 23.

The two plates 37, 38 are superimposed for economy of space, and the apparatus is intended to be placed in an incubator.

According to a particular embodiment, the upper plate 38 includes two compartments 39, 40 for each of the collection 23 and culture medium 20 containers.

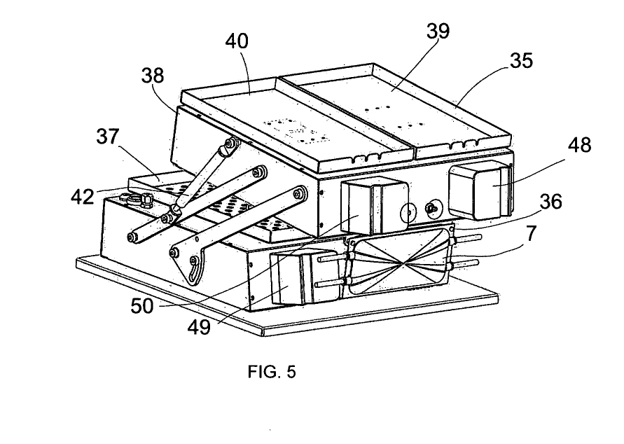

To facilitate the positioning of the kit on the apparatus and for ergonomic reasons, the upper plate 38 is capable of being moved with respect to the lower plate 37 between a top position and a bottom position, as shown in FIGS. 4 and 5, respectively.

For example, the apparatus is equipped with two pneumatic springs 41, 42 connected to the plates 37, 38 so as to enable the movement of the upper plate 38 to a lower height.

According to the invention, the apparatus 35 also includes a device for circulating gas in the enrichment container 7. Said device includes a pump (not shown) of which the outlet 44 is placed in fluidic communication with the gas supply opening 10 of the enrichment container 7 and the inlet 43 is in fluidic communication with a gas source.

The outlet 44 of the pump is placed in communication by means of the fifth tubing 29 with the gas supply opening 10 of the enrichment container 7.

The device for circulating gas enables gas to be introduced in the enrichment container 7 with a flow arranged so as to enable a gas exchange between the culture medium volume and the gas volume at the free interface thereof.

In particular, the pump is a membrane pump. The membrane pump has the advantage of adapting the gas flow rate to the internal pressure of the enrichment bag 7. Indeed, for these pumps, the flow rate is inversely proportional to the pressure. Thus, when the pressure inside the enrichment bag 7 exceeds a certain threshold, the pump stops pumping and therefore protects the system from overpressure.

In addition, the amount of gas or oxygen dissolved in the medium is directly dependent on the pressure exerted on the free contact interface between the gas and the culture medium in the enrichment container 7. The choice of the flow rate/pressure ratio of the pump, the gas volume/medium volume ratio and the pressure drops at the outlet of the enrichment container 7 enables the amount of gas dissolved in the culture medium for a given kit to be adjusted.

According to a particular embodiment, the outlet 44 of the pump is equipped with means for filtration F of the gas stream. Advantageously, the filtration means F are constituted by a sterilising filter 46 having a porosity of less than 2 μm, and in particular around 1.2 μm. This filter makes it possible to avoid the introduction of contaminants into the kit. In addition, this sterilising outlet filter protects the operator.

The inlet 43 of the pump is connected to a gas source such as a gas bottle. Advantageously, the gas source is the ambient air and the inlet 43 of the pump is equipped with means for filtration F of the gas stream enabling to avoid the introduction of particles into the pump, then into the kit. For example, the filtration means are constituted by a sterilising filter 45 with a porosity of less than 2 μm, and in particular around 1.2 μm.

Thus, the pump pressurises the gas and introduces it through the fifth tubing 29 into the enrichment container 7 via the gas supply opening 10, which undergoes a first pressure drop. The gas stream passes through the enrichment container 7 and leaves the container through the gas withdrawal opening 11. The gas then flows along the sixth tubing 31, terminated by a porous filter 32 that causes a sudden narrowing and a second pressure drop. With this latter pressure drop, the gas volume inside the enrichment container 7 is subjected to an overpressure.

The apparatus 35 also includes a device for circulating the culture medium stream between the enrichment container 7, the culture container 1, a possible collection container 23 and a possible culture medium source 20.

This circulation device includes a set of four pumps 47, 48, 49, 50 such as peristaltic pumps each including a pump head intended to receive a first tubing portion 18 and second tubing portion 19, and two third tubing portions 24, upstream and downstream of the connection 26 with the fourth tubing 25, respectively.

The apparatus also includes a control and/or regulation unit for the gas circulation device and the culture medium stream circulation device.

The control and/or regulation unit uses measurements to adjust the pump commands.

Scales are used to determine the weight of at least one container. In particular, each of the receiving means and the attachment means are equipped with a scale so that the weight of each container can be determined at all times.

Alternatively, flow rate measurement means are used to measure the medium supply and medium withdrawal flow rates.

Alternatively, level and/or pressure detection means are used to measure the amount of medium present in the enrichment 7 and culture 1 containers.

These measurements can make it possible to detect the presence of any leakages of medium by means of the control and/or regulation unit via an algorithm for monitoring the balance of material between the containers. A regulation algorithm controls the volume present in the containers and in particular adapts the flow rate of the four medium exchange pumps in order to preserve a volume and a constant exchange.

In addition, this control and/or regulation unit, associated with the arrangement in the vertical plane of the enrichment container, makes it possible to prevent the culture medium from leaving through one of the gas supply 10 and withdrawal 11 openings of the enrichment container 7.

A device for cooling the apparatus, for example by circulation of a heat transfer fluid, can be provided in order to prevent the thermal environment of the cells from being disrupted.

The apparatus is intended to be capable of being placed in an incubator enabling the temperature and the oxygen and carbon dioxide concentration of the environment of the kit to be regulated.

Alternatively, the apparatus also includes internal gas concentration and temperature regulation means. In this case, it is not necessary to place the apparatus in an incubator.

The apparatus is intended to operate without the assistance and monitoring of an operator. An interface between the operator and the software on-board the apparatus is provided. This interface may be integrated in the apparatus or preferably separate from the apparatus so as to be capable of controlling it from outside the incubator and/or the cell culture area. Advantageously, this interface may be a supervisor capable of high-level functions such as data display, the development of a traceability file or functions related to maintenance of the apparatus.

The apparatus is intended to be capable of transmitting an alert in the event of a failure. This alert may be transmitted over a suitable network, for example by means of dry contact.

An example of the implementation of the method of the invention using the kit of FIG. 2 and the apparatus as described above will now be described.

The apparatus is turned on and connected to a computer acting as a supervisor.

The kit of FIG. 2 is removed from its sterile packaging under a suction hood so as to maintain the sterility of the system. All of the clamps and three-way valves are closed. A bag of fresh medium 20 is connected to the kit via the perforator 21. Then the cells to be cultivated are inserted with a small amount of culture medium into the culture bag 1 by means of a syringe, using the three-way valve 33 placed near the medium supply opening 2 of the culture bag 1. The valve 33 is closed back up 33. Then, the air filter is connected to the male Luer 30 at the gas supply opening 10 of the enrichment bag 7.

All of these steps are performed under a suction hood. The kit now forms a closed system and it may be removed from the hood without risking contaminating the cells.

The kit is arranged on the apparatus, beginning with the fresh medium bag 20 on the upper plate 38 and the culture bag 1 on the lower plate 37. Finally, the receiving bag 23 is positioned on the upper plate 38 and the enrichment bag 7 is suspended by inserting the hooks 36 of the apparatus into the holes 14, 15 of the bag. The first, second and third tubings 18, 19, 24 are inserted into the corresponding pump heads of the four peristaltic pumps 47, 48, 49, 50 so as to ensure the circulation of the medium in the kit.

For the circulation of the air, the filter 46 is positioned on the apparatus so as to connect it to the membrane pump integrated in the apparatus.

All of the clamps are opened and the culture medium circulation may begin. The apparatus is disconnected from the computer and is placed in an incubator. It now functions autonomously according to four main modes:

### Filling Mode

In filling mode, the culture bag 1 is filled to a nominal volume, for example around 200 ml, of culture medium, and the enrichment bag 7 is filled to a nominal volume, for example around 100 ml, of culture medium. When the bags 1, 7 have reached the predetermined volume, the apparatus then changes to supply mode.

### Supply Mode

In this mode, the culture container 1 is supplied with fresh medium coming from the fresh medium bag 20 via the enrichment bag 7. This mode continues until the fresh medium volume is insufficient or until it is changed manually to the next mode. The culture medium, after passing into the culture bag 1, is sent into the collection bag 23. The cells in the culture bag 1 are either adherent or suspended. In this case, they must be heavy enough to be held in the container and not be driven by circulation of the culture medium.

The flow rate of the culture medium is attached so as, for example, to supply the culture bag 1 with fresh medium for around 24 hours, for example. In particular, it is less than 1 ml/min. This flow rate is set so as to ensure good preservation/expansion of the cells and maintain a correct culture medium cost.

### Recycling Mode

In this embodiment, the used medium, i.e. coming from the culture bag 1, is recycled via the enrichment bag 7. The recirculation or the recycling is performed for example for hours. In this mode, the flow rate of medium may be greater than 1 ml/min, for example on the order of 6 ml/min.

To perform sampling during culturing, the apparatus is equipped with a pause button.

By varying the flow rates of the pumps, it is possible to combine the supply mode and the recycling mode. The volumes of discarded medium and fresh medium are then regulated so as to maintain the stable system.

### Drainage Mode

In this mode, the culture medium bag 1 is drained, for example of 120 ml of medium. The cells are collected by means of a syringe connected to the three-way valve 34 located near the medium withdrawal opening 3 of the culture bag 1. To do this, the culture bag 1 is detached from the rest of the kit by welding below the three-way valves 34 and the cells are collected under a suction hood.

The apparatus is programmed to change automatically from one mode to another without intervention by the user. The apparatus is therefore totally autonomous. It is obvious that, at any time, the user can intervene to manually change modes.

1-15. (canceled)

16. Method for expanding and/or preserving cells inside a culture container containing a culture medium, said method comprising renewing said culture medium by means of a stream of culture medium of which at least a portion comes from a culture medium volume contained in a container for gas enrichment of said culture medium, said enrichment container also containing a gas volume that is separated from the culture medium volume by a free interface, and renewing the gas volume by means of a gas stream that is introduced into the enrichment container directly in the gas volume, with the flow of said stream being arranged so as to enable a gas exchange between said culture medium volume and said gas volume at the interface thereof.

17. Method according to claim 16, further comprising arranging the flow of the gas stream so that the pressure of the gas volume is greater than ambient pressure.

18. Method according to claim 17, comprising flowing the gas stream in a path having a portion for supplying gas to the container and a portion for removing gas from said container, introducing the gas under pressure into the container by said supply portion, and the withdrawal portion has a pressure drop that is arranged so as to maintain a pressure greater than the ambient pressure in the enrichment container.

19. Method according to claim 18, comprising circulating the gas stream in the supply portion by means of at least one pump or a pressurised tank.

20. Method according to claim 16, comprising supplying the enrichment container with a stream of culture medium.

21. Method according to claim 20, wherein the culture medium stream supplying the enrichment container comes at least partially from the culture container and/or a fresh culture medium source.

22. Method according to claim 16, comprising withdrawing a stream of culture medium from the culture container so as to be stored.

23. Kit for implementing the method according claim 16, including a culture container intended to contain cells in a culture medium and an enrichment container for gas enrichment of the culture medium, said enrichment container having a supply opening for the culture medium to be enriched and a withdrawal opening for the enriched culture medium, said withdrawal opening is in fluidic communication with a supply opening of the culture medium, said enrichment container also having a supply opening and a withdrawal opening for gas, in which said openings of the enrichment container are arranged so that said container can contain a culture medium volume into which the culture medium supply and withdrawal openings lead, and a gas volume into which the gas supply and withdrawal openings lead.

24. Kit according to claim 23, wherein the enrichment container includes a flexible envelope defining a variable internal volume into which the openings lead.

25. Kit according to claim 23, wherein the enrichment container is equipped with suspension means enabling said container with the gas volume to be arranged above the culture medium volume.

26. Kit according to claim 23, wherein the culture medium supply and gas withdrawal openings are arranged on one side of the enrichment container, and the gas supply and culture medium withdrawal openings are arranged on an opposite side of said enrichment container.

27. Kit according to claim 23, wherein the culture container includes at least one deflector arranged on a direct flow path between the culture medium supply opening and a withdrawal opening of said culture container.

28. Apparatus in which a kit according to claim 23 is intended to be used, said apparatus including:
means for attaching an enrichment container by arranging a gas volume above a culture medium volume;
means for receiving a culture container, an optimal collection container and an optimal culture medium source;
a device for circulating gas contained in the enrichment container, said device including a pump of which an outlet is placed in communication with a gas supply opening of the enrichment container and an inlet is in communication with a gas source, said device enabling gas to be introduced into the enrichment container with a flow arranged so as to enable a gas exchange between said culture medium volume and said gas volume at the interface thereof; and
a device for circulating the culture medium stream between the enrichment container, the culture container, said optimal collection container and said optimal culture medium source.

29. Apparatus according to claim 28, further comprising a unit for control and/or regulation of the device for circulating the gas and the device for circulating the stream of culture medium.

30. Apparatus according to claim 29, wherein the control and/or regulation unit includes scales for determining weight of at least one container.
